(19) United States
(12) Patent Application Publication
(10) Pub. No.: US 2018/0172666 A1
(43) Pub. Date: Jun. 21, 2018

(54) HYDROGEL-BASED MICROFLUIDIC CHIP FOR CO-CULTURING CELLS

(71) Applicant: SOGANG UNIVERSITY RESEARCH FOUNDATION, Seoul (KR)

(72) Inventors: Bong Geun CHUNG, Gwacheon-si (KR); Jong Min LEE, Seoul (KR); Hye In SEO, Seongnam-si (KR); Jun-Hyuk BAE, Goyang-si (KR)

(21) Appl. No.: 15/557,224

(22) PCT Filed: Jul. 21, 2015
(86) PCT No.: PCT/KR2015/007552

§ 371 Date: Sep. 11, 2017

(30) Foreign Application Priority Data
Mar. 11, 2015 (KR) 10-2015-0033951

(51) Int. Cl. G01N 33/50; G01N 21/17; B01L 3/00; C12M 3/00; G01N 33/574; C12M 3/06

(52) U.S. Cl. CPC G01N 33/5005; G01N 21/17; B01L 3/502715; C12M 3/00; C12N 2513/00; C12M 23/16; G01N 2021/1714; C12N 2502/30; C12N 2502/28; G01N 33/574

(57) ABSTRACT

Provided are a hydrogel-based microfluidic chip for cell co-culture and a use thereof, wherein the microfluidic chip allows the co-culture of cancer cells and vascular endothelial cells; can be widely applied in various studies associated with cancer; is suitable in studies on the photothermal therapy effect on, especially, cancer cells; and has excellent biocompatibility, mechanical properties, and economical feasibility.

### TECHNICAL FIELD

The present invention relates to a hydrogel-based microfluidic chip for co-culturing cells.

### BACKGROUND ART

Glioblastoma is one of the most common forms of brain tumor and malignant tumor, and it is known that glioblastoma is highly unlikely to be cured as compared with its outbreak frequency. There are various methods of tumor treatment, such as radiotherapy or chemotherapy, but it is very important to search for safe treatment methods in consideration of side effects [1, 2]. Glioblastoma is highly resistant to radiotherapy and chemotherapy, and the treatment methods, such as anti-angiogenesis and apoptosis, have limitations [3]. Meanwhile, breast cancer that frequently occurs in women is treatable only by resection in cases of early detection, but the breast cancer has a heavy mortality if its metastasis starts. Cancer may metastasize to various parts of the body, and the metastasized cancer is difficult to find, resulting in death, and thus it is very important to understand this mechanism. Therefore, various levels of studies associated with metastasis of cancer cells, including regulation of gene expression, and signaling, etc., are needed. Cancer metastasis occurs through cell migration, intravasation, extravasation, delivery, and the like. Researches was carried out that genes play an important role in the metastasis stage. Researches were carried out about genes performing an extravasation role of passing through microvessels of other organs and about genes involved in various metastasis procedures [4, 5]. Researches were carried out about the brain tumor therapy effect by the induction of differentiation into bone morphogenetic proteins (BMP) using CD133+ tumor stem cells. However, the method of inhibiting self-renewal using tumor stem cells may be used as a novel concept of treatment method, but necessary tumor stem cells existing in small amounts make researches difficult.

Recently, a lot of researches have been carried out about photothermal therapy for cancer treatment. In the photothermal therapy, cancer cells or cancer tissues are damaged by heat converted from near-infrared ray light, and gold nanoparticles may be used as an excellent photothermal agent. Gold nanoparticles are favorably biocompatible and easily surface-transformed, and thus are easily combined with biopolymers, antibodies, DNA, and the like. In addition, gold nanoparticles can control the surface plasmon resonance effect according to the shape and size thereof. Especially, gold nanorods exhibit surface plasmon resonance effects at two wavelengths due to the anisotropic shape thereof. They are horizontal-axis surface plasmon resonance at a wavelength of 520 nm, corresponding to the area of the gold nanorod, and vertical-axis surface plasmon resonance at a wavelength of 650-900 nm (near-infrared wavelength), and especially, the gold nanorod exhibits a strong absorption at the near-infrared wavelength. Here, the aspect ratio may be controlled by shifting the wavelength region of the vertical-axis surface plasmon resonance [10].

When gold nanorods are injected into cancer tissues and the long wavelength near-infrared light is irradiated thereto, the gold nanorods absorb energy to generate heat in only the cancer tissues restrictively, and thus the gold nanorods deeply (˜10 cm) permeate into the cancer tissues without the damage to normal tissues, leading to a photothermal effect [11, 12]. Recently, gold nanorods conjugated to a biopolymer, such as polyethyleneglycol (PEG) or a biopolymer, is mostly used in the photothermal therapy. PEG can prevent the coagulation of nanoparticles and the adsorption of non-specific proteins, and can stay in the blood for a long period of time, and thus can help the accumulation of nanoparticles in cancer cells [13]. Silica can be effectively used as a drug delivery system for drug delivery. There is a limit in loading a drug on a surface of the gold nanorod, and thus, when the gold nanorod is coated with silica nanoparticles and a drug is loaded thereon, photothermal therapy and chemotherapy may be performed in combination [14]. As described above, gold nanorods are being mostly researched as a photothermal agent for thermal therapy and actively researched for various biomedical applications due to the above-described distinctive optical characteristics thereof.

Researches on microfluidic chips that can control microenvironments surrounding cells were previously carried out [15, 16]. The change of the microenvironments can contribute to cancer growth and proliferation. In a case where a microfluidic chip is applied to cancer cells, various phenomena occurring in the human body, such as angiogenesis, immune response, and cancer metastasis, can be observed, and intercellular interactions and interactions between cells and the cellular matrix can be observed. Thus, systematic studies and in vitro drug and toxicity evaluation can be carried out. Recently, microchips for isolating cancer cells from peripheral blood were developed [17]. Circulating tumor cells in the blood are origins of cancer metastasis. It is very difficult to isolate these cells from cancer patients, but the circulating tumor cells were effectively isolated using microchips. In addition, besides the technique of isolating cancer cells using an antigen-antibody interaction, techniques of continuously isolating circulating tumor cells from breast cancer patients using hydrodynamic characteristics, such as size and density of cancer cells, were developed [18]. These techniques allow the isolation of various kinds of circulating tumor cells, and thus can be applied to various cells. However, the metastasis and treatment of cancer cells were not considered in these microchips for detecting circulating tumor cells. For precise simulation and control of tumors and surrounding microenvironments thereof, a three-dimensional co-culture with immune cells, endothelial cells, and fibroblasts as well as cancer cells is required. This study requires an organic fusion of engineering research and cancer-related pathological knowledge. Drug effects and pharmacokinetics of 5-fluorouracil as an anticancer drug were analyzed by culturing liver, cancer cells, and marrow cells in a hydrogel-based microfluidic chamber [19]. This microfluidic chip enables high-throughput screening in toxicity evaluation. In addition, microfluidic devices for three-dimensional cell culture and analysis were developed [20]. Vascular endothelial cells were cultured to form a three-dimensional vascular structure in a channel, and then an angiogenesis reaction was investigated. When vascular endothelial cells and smooth muscle cells were co-cultured, the influence of the smooth muscle cells on the angiogenesis reaction of the vascular endothelial cells could be observed, and three-dimensional culture of breast cancer cells were researched [21]. The microfluidic chip cultured in a three-dimensional manner can simulate various human body environments, leading to precise analysis. However, the existing microfluidic chips did not effectively consider the photothermal therapy study and metastasis study through various sections. The currently developed hydrogel-based microfluidic chip implements a three-dimensional phenomenon to vary physical and chemical mechanisms of cancer cells, and thus can be utilized in new drug developments or drug evaluations. Therefore, the hydrogel-based microfluidic chip for co-culture can be used as a very potential device for studies of photothermal therapy and metastasis of cancers.

Throughout the entire specification, many papers and patent documents are referenced and their citations are represented. The disclosure of cited papers and patent documents is entirely incorporated by reference into the present specification, and the level of the technical field within which the present invention falls and details of the present invention are explained more clearly.

### DETAILED DESCRIPTION OF THE INVENTION

### Technical Problem

The present inventors have endeavored to develop a microfluidic chip for cell co-culture, capable of efficiently co-culturing, especially, cells. As a result, the present inventors have developed a microfluidic chip, which allows independent culture of cancer cells in respective chambers and co-culture of cancer cells and vascular endothelial cells by fabricating the microfluidic chip including microchambers, bridge channels, and a microfluidic channel, and injecting gelatin hydrogels and vascular endothelial cells through the microfluidic channel to construct a barrier, to thereby suppress a molecular diffusion between the microchambers, and thus the present inventors have completed the present invention.

Therefore, an aspect of the present invention is to provide a hydrogel-based microfluidic chip for cell co-culture.

Another aspect of the present invention is to provide a method for cell co-culture using the microfluidic chip of the present invention.

Still another aspect of the present invention is to provide a method for analyzing a photothermal therapy effect on cancer cells using the microfluidic chip of the present invention.

Other purposes and advantages of the present disclosure will become more obvious with the following detailed description of the invention, claims, and drawings.

### Technical Solution

In order to accomplish these objects, there is provided a microfluidic chip for co-culture of cancer cells, including: (a) one or more microchambers as cell culture sections, including sample inlets; (b) bridge channels connected to the microchambers; and (c) a microfluidic channel connected to the bridge channels and including a hydrogel inlet, wherein a barrier is formed by hydrogels, in which gelatin and an acryl polymer are mixed, and vascular endothelial cells, the hydrogels and the vascular endothelial cells being injected through the hydrogel inlet.

The present inventors have endeavored to develop a microfluidic chip for cell co-culture, capable of efficiently co-culturing, especially, cells. As a result, the present inventors have developed a microfluidic chip, which allows independent culture of cancer cells in respective chambers and co-culture of cancer cells and vascular endothelial cells by fabricating the microfluidic chip including microchambers, bridge channels, and a microfluidic channel, and injecting gelatin hydrogels and vascular endothelial cells through the microfluidic channel to construct a barrier, to thereby suppress a molecular diffusion between the microchambers.

The main feature of the present invention is that a barrier composed of hydrogels and vascular endothelial cells is placed in the microfluidic chip for co-culture of cancer cells, thereby suppressing the molecular diffusion between co-cultured cancer cells. In addition, the bridge channels filled with hydrogels and vascular endothelial cells are connected to the microchambers for culturing cancer cells, thereby allowing the co-culture of cancer cells and vascular endothelial cells. The co-culture of cancer cells and vascular endothelial cells can be widely applied in various studies associated with cancer. It was actually verified that cancer cells migrated toward vascular endothelial cells when the cancer cells and the vascular endothelial cells were cultured by using the microfluidic chip of the present invention.

In the hydrogel-based microfluidic chip for cell co-culture of the present invention, the microchambers correspond to cell culture sections, and include sample inlets. Through the sample inlets, cells, a cell culture medium, a sample necessary for analysis, nanoparticles exhibiting a photothermal effect, and the like may be injected.

According to an embodiment of the present invention, one or more microchambers are formed in the microfluidic chip for cell co-culture of the present invention, and are arranged in one or more columns and one or more rows. Most preferably, the microchambers are arranged in two columns and two rows in the microfluidic chip for cell co-culture of the present invention.

In the microfluidic chip for cell co-culture of the present invention, the microchambers are connected to the bridge channels.

According to an embodiment of the present invention, in the microfluidic chip for cell co-culture of the present invention, the microchambers, the bridge channels, and the microfluidic channel have a thicknesses of 200-300 μm, 30-50 μm, and 200-300 μm, respectively, and thus the microchambers and the bridge channels, which are connected to each other, and the bridge channels and the microfluidic channels, which are connected to each other, form a step difference.

In the microfluidic chip for cell co-culture of the present invention, the bridge channels are connected to the microfluidic channel. The microfluidic channel is disposed such that it is connected to the microchambers through the bridge channels, and preferably has a cruciform.

In the microfluidic chip for the cell co-culture of the present invention, the molecular diffusion between the microchambers is suppressed by the barrier, which is formed of hydrogels, in which gelatin and an acryl polymer are mixed, and the vascular endothelial cells, which are injected through the hydrogel inlet, thereby allowing independent cell culture in the respective microchambers.

According to an embodiment of the present invention, the acryl polymer is selected from the group consisting of a methacrylate copolymer, a methyl methacrylate polymer, an acrylate and methacrylate copolymer, an ethoxyethyl methacrylate copolymer, a cyanoethyl methacrylate copolymer, an aminoalkyl methacrylate copolymer, a poly(acrylate) copolymer, a polyacrylamide copolymer, a glycidyl methacrylate copolymer, and a mixture thereof. According to another embodiment of the present invention, the acryl polymer is selected from the group consisting of a methacrylate copolymer, a methyl methacrylate polymer, an acrylate and methacrylate copolymer, and a mixture thereof. According to a specific embodiment of the present invention, the acryl polymer is a methacrylate copolymer.

The molecular diffusion of the cell co-culture can be controlled by adjusting the concentration of the hydrogels, in which gelatin and an acryl polymer are mixed. The hydrogels in which, gelatin and an acryl polymer are mixed, may be prepared by various methods known in the art. For example, gelatin is mixed and stirred in phosphate buffered saline (PBS) at 50° C. until the gelatin is completely dissolved in the PBS, and methacrylic anhydride is added at a rate of 0.5 ml/min, thereby preparing gelatin methacrylate (GelMA) hydrogels.

According to an embodiment of the present invention, the hydrogels, in which gelatin and an acryl polymer are mixed, have a concentration of 5-15 w/v %, more preferably, 7-12 w/v %, and most preferably 10 w/v %.

In the hydrogel-based microfluidic chip for cell co-culture of the present invention, the hydrogels, in which gelatin and an acryl polymer are mixed, are photo-crosslinked.

As used herein, the term “photo-crosslinking” refers to a polymerization through covalent and physical cross-linkage formed by irradiating light in the presence of a photoinitiator. The photoinitiator is a chemical material and initiates a polymerization reaction and/or a radical crosslinkage by light.

For the photo-crosslinkage of the hydrogels, in which gelatin and an acryl polymer are mixed, of the present invention, GelMA hydrogels are mixed with PBS and 2-hydroxy-1-(4-(hydroxyethoxy)phenyl)-2-methyl-1-propanone, as a photoinitiator, at 80° C., and then the mixture is injected into chambers, and subjected to UV (360-480 nm wavelength) irradiation to induce the photo-crosslinkage.

According to an embodiment of the present invention, in the hydrogel-based microfluidic chip for cell co-culture, the hydrogels, in which gelatin and an acryl polymer are mixed, are encapsulated.

As used herein, the term “encapsulation” refers to an immobilization of cells in a semi-permeable gel (or membrane), which is polymerized to allow the bidirectional diffusion of molecules, such as an inflow of oxygen, nutrition, and growth factors, which are necessary for cell metabolism, and an outflow of wastes and therapeutic proteins. The main motive of cell encapsulation is to solve the problems in graft rejection at the application to tissue engineering to reduce the long-term use of immunosuppressive drugs for preventing side effects after organ transplantation.

In order to simulate the vascular structure in the microfluidic chip so as to use the microfluidic chip of the present invention in studies on the relationship between cancer metastasis and blood vessels, vascular endothelial cells were encapsulated before use.

The microfluidic chip of the present invention is fabricated by using a polymer material selected from the group consisting of poly(dimethylsiloxane) (PDMS), polymethylmethacrylate (PMMA), polyacrylates, polycarbonates, polycyclic olefins, polyimides, and polyurethanes. Most preferably, the microfluidic chip of the present invention is fabricated by using poly(dimethylsiloxane) (PDMS).

The microfluidic chip of the present invention is joined to an upper portion of a plate facilitating optical measurement, which is selected from the group consisting of slide glass, crystal, and glass. Most preferably, the microfluidic chip of the present invention is joined to an upper portion of the glass.

Examples of the cancer cells that can be cultured in the microfluidic chip for cell co-culture of the present invention may include, but are not particularly limited to, breast cancer cells, brain tumor cells, prostate cancer cells, rectal cancer cells, lung cancer cells, pancreatic cancer cells, ovarian cancer cells, bladder cancer cells, endometrial cancer cells, cervical cancer cells, liver cancer cells, kidney cancer cells, thyroid cancer cells, bone cancer cells, lymphoma cancer cells, or skin cancer cells.

In accordance with another aspect of the present invention, there is provided a method for cell co-culture, including:
(a) preparing a microfluidic chip for cell co-culture, comprising: (i) one or more microchambers as cell culture sections, including sample inlets; (ii) bridge channels connected to the microchambers; and (iii) a microfluidic channel connected to the bridge channels and including a hydrogel inlet;
(b) injecting hydrogels, in which gelatin and an acryl polymer are mixed, and vascular endothelial cells into the hydrogel inlet, and then inducing photo-crosslinking to construct a barrier; and
(c) injecting cancer cells into the sample inlets, followed by culturing.

Since the method for cell co-culture of the present invention is directed to culturing of cancer cells and vascular endothelial cells using the above-described microfluidic chip for cell co-culture, descriptions of overlapping contents therebetween are omitted to avoid excessive complexity of the present specification.

In accordance with another aspect of the present invention, there is provided a method for analyzing a photothermal therapy effect on cancer cells, the method including:
(a) preparing a microfluidic chip for cell co-culture, comprising: (i) one or more microchambers as cell culture sections, including sample inlets; (ii) bridge channels connected to the microchambers; and (iii) a microfluidic channel connected to the bridge channels and including a hydrogel inlet;
(b) injecting hydrogels, in which gelatin and an acryl polymer are mixed, and vascular endothelial cells into the hydrogel inlet, and then inducing photo-crosslinking to construct a barrier;
(c) injecting cancer cells through the sample inlets, followed by culturing;
(d) injecting nanoparticles exhibiting a photothermal effect through the sample inlets, followed by culturing; and
(e) irradiating a laser to the microchambers to analyze the extent of survival or death of the cancer cells.

As used herein, the term “photothermal therapy” (photothermal radiation or optical thermal warmth) refers to a treatment of solid tumors, and typically includes a step of converting absorbed light into local heat through a non-radioactive mechanism. Near-infrared rays (NIR) used in photothermal therapy can deeply penetrate into tissues with high spatial precision without damage to general biological tissues due to a low near-infrared absorption into general tissues.

According to an embodiment of the present invention, the photothermal therapy effect of nanoparticles is analyzed by culturing cancer cells in the microfluidic chip for co-culture of cancer cells of the present invention, injecting nanoparticles exhibiting a photothermal effect into each microchamber, irradiating a laser thereto, and then analyzing the degree of survival or death of cancer cells.

According to an embodiment of the present invention, the nanoparticles used in the analysis of the cancer cell photothermal effect are gold nanorods.

### Advantageous Effects

Features and advantages of the present invention are summarized as follows.

(a) The present invention provides a hydrogel-based microfluidic chip for cell co-culture and a use thereof.

(b) The microfluidic chip of the present invention, which allows the co-culture of vascular endothelial cells and cancer cells, can be widely used in cancer-related studies, and is suitable for the photothermal therapy effect on, especially, cancer cells.

(c) The microfluidic chip of the present invention has excellent biocompatibility, mechanical properties, and economical feasibility.

### BRIEF DESCRIPTION OF THE DRAWINGS

FIG. 1 shows a gelatin methacrylate hydrogel-based microfluidic chip for co-culture: (A) Schematic diagram of gelatin methacrylate hydrogel-based microfluidic chip for co-culture, including a microfluidic channel and microchambers; and (B) Image of gelatin methacrylate hydrogel-based microfluidic chip for co-culture;

FIGS. 2A to 2C show SEM images of 5 w/v %, 15 w/v %, and 25 w/v % photo-crosslinkable GelMA hydrogels. Scale bars indicate 20 μm;

FIGS. 3A and 3B show effects of GelMA hydrogel concentrations (5-25 w/v %). FIGS. 3A and 3B show pore size and aspect ratio, respectively. The aspect ratio means the value of the length of pores divided by width of pores (*p<0.05, **p<0.01);

FIG. 4 shows analysis results of 10 w/v % gelatin methacrylate hydrogels for barrier and cell encapsulation: (A) SEM image of 10 w/v % gelatin methacrylate hydrogels; (B) fluorescent images of molecular diffusion of four square-shaped microchambers (Left-up (LU), Right-up (RU), Left-down (LD), and Right-Down (RD)). Rhodamine B-dextran was only injected into RU microchamber, and was diffused to LD microchamber. (C) Analysis graph of the molecular diffusion through 10 w/v % gelatin methacrylate hydrogels for 1 day and 5 days;

FIG. 5 shows synthesis results of gold nanorods. (A) TEM image of synthesized gold nanorods; (B) UV-visible spectrum results of gold nanorods stabilized with CTAB; and (C) Schematic diagram of injection of synthesized gold nanoparticles into square-shaped microchambers;

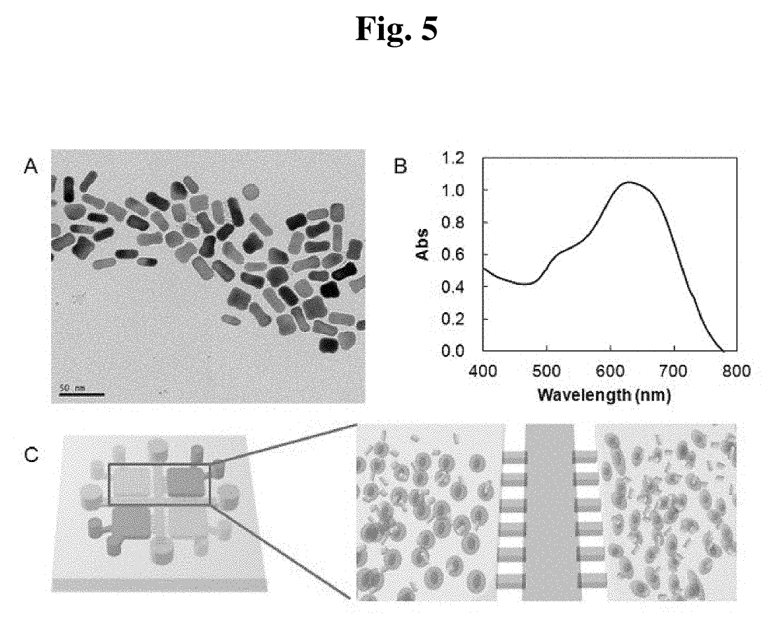

FIG. 6 shows analysis results of photothermal therapy effect of gold nanorods. (A) Analysis of temperature increase depending on gold nanorod concentration after NIR laser irradiation (808 nm, 7 W); (B) CCK-8 live/dead assay graphs of photothermal therapy effects on glioblastoma cells and breast cancer cells in 96-well plate; and (C) live/dead assay fluorescent images of glioblastoma cells and breast cancer cells in co-culture microfluidic chip; and

FIG. 7 shows confocal microscopic images with respect to metastasis of cancer cells. (A) Schematic diagram of hydrogel-based co-culture microfluidic chip for study of cancer cell metastasis; (B) Confocal microscopic image of MCF7 cells; (C) Confocal microscopic image of U87MG cells on glass substrate; (D) Confocal microscopic image of U87MG cells metastasized to GelMA barrier from chamber in device; (E) Confocal microscopic image of GelMA barrier chamber containing metastatic U87MG cells; (F) Confocal microscopic image of MCF7 cells cultured in chamber; and (G) High-magnification confocal microscopic image of bridge channel containing U87MG cells metastasized to GelMA barrier from chamber in device.

### MODE FOR CARRYING OUT THE INVENTION

Hereinafter, the present invention will be described in detail with reference to examples. These examples are only for illustrating the present invention more specifically, and it will be apparent to those skilled in the art that the scope of the present invention is not limited by these examples.

### EXAMPLES

### Materials and Methods

### Fabrication of 3D Microfluidic Co-Culture Device

The microchambers and bridge channels were manufactured by two-step photolithography methods known in the art. To fabricate 3D microfluidic co-culture device, microchambers and bridge channels were designed by AutoCAD program. To manufacture bridge channels, SU-8 25 photoresist was spin-coated on a silicon wafer (1,000 rpm, 60 s, and 40 m in thickness). To manufacture microchambers, SU-8 100 was spin-coated on SU-8 25 photoresist-patterned substrates (3,000 rpm, 60 s, and 250 m in thickness). The poly(dimethylsiloxane) (PDMS) precursor solution was molded from the photoresist-patterned silicon wafer, and PDMS-based 3D microfluidic culture device was bonded into glass slides using oxygen plasma treatment (Femto Science, Korea).

The microfluidic chip including four square-shaped microchambers (Left-up (LU), Right-up (RU), Left-down (LD), and Right-Down (RD)) and a cruciform microfluidic channel connected to bridge microfluidic channels. The four square-shaped microchambers (250 μm in thickness) are connected by the bridge microchannels (40 μm in thickness), and the bridge microchannels are connected with the cruciform microfluidic channel (250 μm in thickness). The cruciform microfluidic channel was manufactured in order to prevent the encapsulation of vascular endothelial cells in gelatin methacrylate hydrogels and the molecular diffusion between square-shaped microchambers, and the bridge microchannels were designed to increase the resistance of fluid. Resultantly, the gelatin methacrylate hydrogels were crosslinked by UV light in only the cruciform microfluidic channel, and breast cancer cells and glioblastoma cells were injected across each other in the square microchambers. Then, the molecular diffusion effect of 10 w/v % gelatin methacrylate hydrogels was investigated. By injecting rhodamine B-dextran into RU microchamber, the molecular diffusion of rhodamine B-dextran to LD microchamber was verified, and the molecular diffusion of gelatin methacrylate hydrogels was verified for 1 day and 5 days. Therefore, the gelatin methacrylate hydrogels were used for cell encapsulation and a barrier in the cruciform microfluidic channel.

### Gelatin Methacrylate (GelMA) Hydrogels Synthesis

For the photo-crosslinkable GelMA hydrogels, type A porcine skin gelatin was stirred at 50° C. and phosphate buffered saline (PBS, GIBCO, USA) was mixed until fully dissolved. Methacrylic anhydride was added at a rate of 0.5 mL/min under stirring conditions for 2 h. The mixture was dialyzed against distilled water using 12-14 kDa-cutoff dialysis tubing for 3-4 days at 40° C. to remove salts and methacrylic acids. The solution was lyophilized for 1 week and was subsequently stored at −80° C.

### Gold Nanorod Synthesis

Gold nanorods were synthesized by the seed-growth method. First, the seed solution was prepared by adding 0.25 mL of 0.01 M aqueous HAuCl₄ solution and 0.6 mL of 0.01 M NaBH4 solution to 7.5 mL of 0.1 M CTAB solution. Here, the seed solution was stabilized at room temperature for 2 hours or longer before use. The growth solution was prepared by adding 0.2 ml of 0.01 M HAuCl₄, 0.03 mL of 0.01 M AgNO3, and 0.032 mL of 1 M ascorbic acid to 4.75 mL of 0.1 M CTAB. 0.01 mL of the prepared seed solution was added to the growth solution, and then the mixture was stabilized at room temperature for 3 hours or longer, thereby synthesizing gold nanorods.

### Scanning Electron Microscope

The structure of GelMA hydrogels was analyzed by using a scanning electron microscope (SEM). The swollen hydrogels were frozen and were subsequently lyophilized. The lyophilized samples were cut and their cross-sections were coated with platinum using a turbo sputter coater (EMITECH, K575X). SEM images were obtained at a high voltage of 30 kV.

### Culture of Cancer Cells

Endothelial cells were cultured together with an endothelial cell culture medium (EGM2+Single Quot Kit Components, Lonza, Switzerland) in flask coated with 2% gelatin, and breast cancer cells (MCF7) and glioblastoma cells (U87MG) were cultured in DMEM supplemented with 10% fetal bovine serum (FBS) and 1% penicillin-streptomycin.

### Loading of GelMA Hydrogels and Cell-Encapsulated Collagen Gels

To culture vascular endothelial cells in a 3D manner, 2×10⁶ cells/mL were suspended and encapsulated within 100 μL of the GelMA hydrogel solution. Of these, 20 μL of endothelial cell-encapsulated GelMA hydrogel solution was injected into the cruciform channel. Through UV irradiation for 20 seconds, the GelMA hydrogels form a barrier in the microfluidic chip by photo-crosslinkage. Then, 2×10⁶ cells/mL of MCF7 cells and U87MG cells were injected into square-shaped LU, RU, RD, and LD chambers, crossing each other, together with 10 μL of culture medium.

### Analysis of Photothermal Therapy Effect

The cells were injected into the chambers, and cultured for 1 day to make cells adhere to the chambers, and 20, 30, and 40 μl of gold nanorods were mixed with 200 μl of the cell culture medium, and the mixture was injected through the chamber inlet, followed by NIR laser irradiation, and then the temperature increase was investigated. In addition, glioblastoma cells and breast cancer cells were cultured in the chip for one day, and in a similar manner, the cells were treated by NIR laser irradiation and analyzed by live/dead assay.

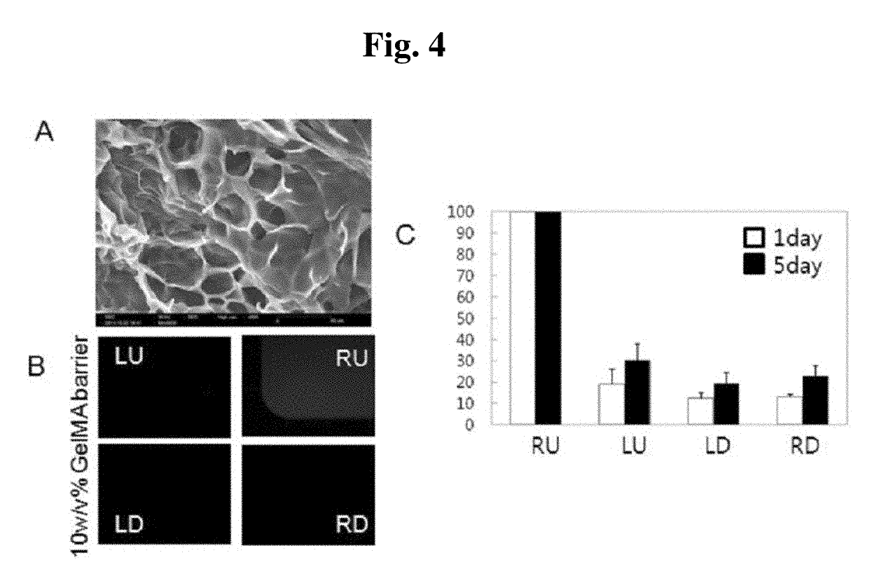

The live/dead assay was carried out through the following method. The breast cancer cells and glioblastoma cells were injected at 1×10⁵ into the 96-well plate and the microchambers. One day after cell injection, the cell culture medium was exchanged with a cell culture medium containing 15 v/v %, and placed for about 6 hours in a cell incubator. Then, NIR was irradiated to the chambers and the 96-well plate. Resultantly, the cell viability was analyzed by CCK-8 (cell-counting kit-8, USA) in the 96-well plate (FIG. 4b), and analyzed through fluorescence using a confocal microscope by live/dead assay (invitrogen, USA) in the microchambers (FIG. 4c).

### Results and Discussion

### Fabrication of GelMA Hydrogel-Based 3D Microfluidic Co-Culture Device

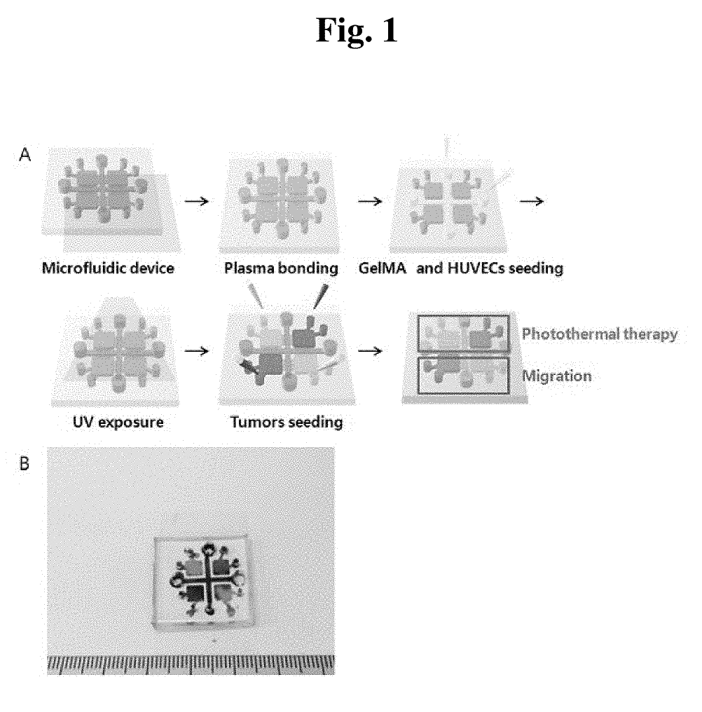

We developed the photo-crosslinkable GelMA hydrogel-based 3D microfluidic culture device (FIG. 1). The GelMA hydrogel-based 3D microfluidic device was fabricated by a two-step photolithography process to be composed of four microchambers and a cruciform microfluidic channel connected to bridge microchannels (FIG. 1C). The four microchambers (250 μm in thickness) were connected by microgrooved bridge microchannels (40 μm in thickness) (FIG. 1C).

The 250 μm-thick microchambers were filled with vascular endothelial cell-encapsulated GelMA hydrogels, breast cancer calls, and glioblastoma cells. The 40 μm-thick microgrooved bridge channels increased the fluidic resistance. GelMA hydrogels were photo-crosslinked via UV in the cruciform microchannel. The cruciform photo-crosslinked GelMA hydrogels in the microchamber function as a physical barrier to inhibit the molecular diffusion across bridge microchannels, thereby allowing culture of vascular endothelial cells. Then, breast cancer cells and glioblastoma cells were injected while crossing each other. This multi-compartment microfluidic culture device has many advantages in cellular interaction and high-throughput drug screening, but in the previous microfluidic co-culture device, the photothermal therapy and the photo-crosslinkable hydrogel-based 3D microfluidic device for co-culture of cancer cells were not considered.

### Effects of GelMA Hydrogel Concentration on Porosity and Molecular Diffusion

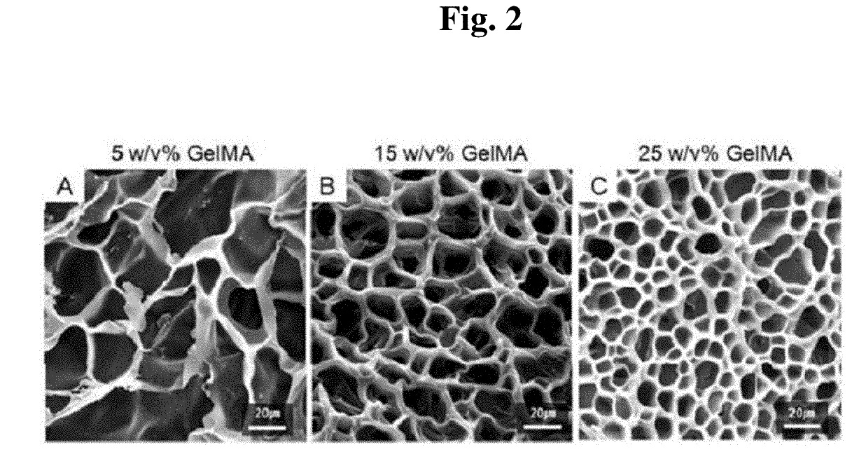

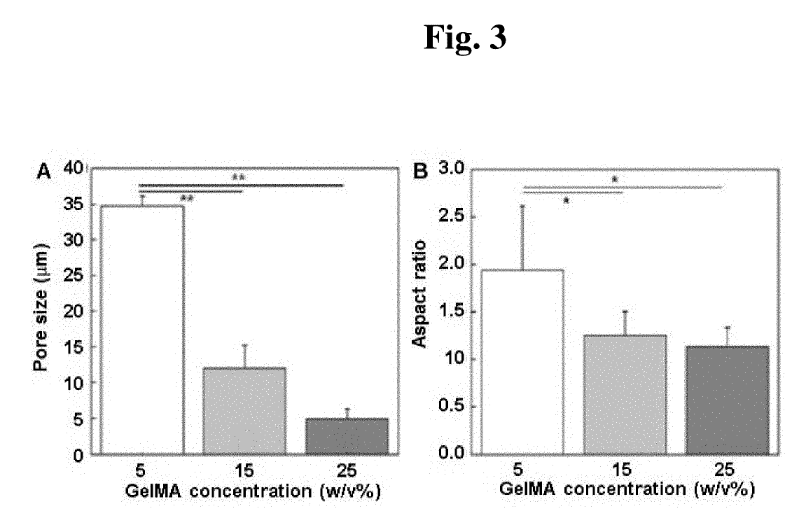

As a result of verifying the effect of GelMA hydrogel concentration on the porosity, the pore size was inversely proportional to GelMA hydrogel concentration (FIG. 2). SEM images indicate that the porosity of 25 w/v % GelMA hydrogels showed uniform sizes and shapes compared to 5 w/v % GelMA hydrogels (FIGS. 2a to 2c). The pore size of 5 w/v % GelMA hydrogels was 34 μm, whereas the pore size of 25 w/v % GelMA hydrogels was 4 μm (FIG. 3A). The porosity of 25 w/v % GelMA hydrogels showed circular shapes (aspect ratio=1), whereas 5 w/v % GelMA hydrogels showed elliptical shapes (aspect ratio=1.9, FIG. 3B). Furthermore, as a result of investigating the effect of GelMA hydrogel concentration on the molecular diffusion, the molecular diffusion easily occurred in 5 w/v % GelMA hydrogels, whereas 25 w/v % GelMA hydrogels completely inhibited the molecular diffusion. Therefore, it was determined that 5 w/v % GelMA hydrogels could not be used as a barrier. In contrast, 15 w/v % GelMA hydrogels were determined to be unfavorable since they may be used as a barrier but the pore size thereof is too small to encapsulate cells. In the present invention, it was determined that the suitable concentration of GelMA hydrogels was 10 w/v % GelMA for the use as a barrier of the microfluidic chip and for cell encapsulation.

### Analysis of Photothermal Therapy Effect

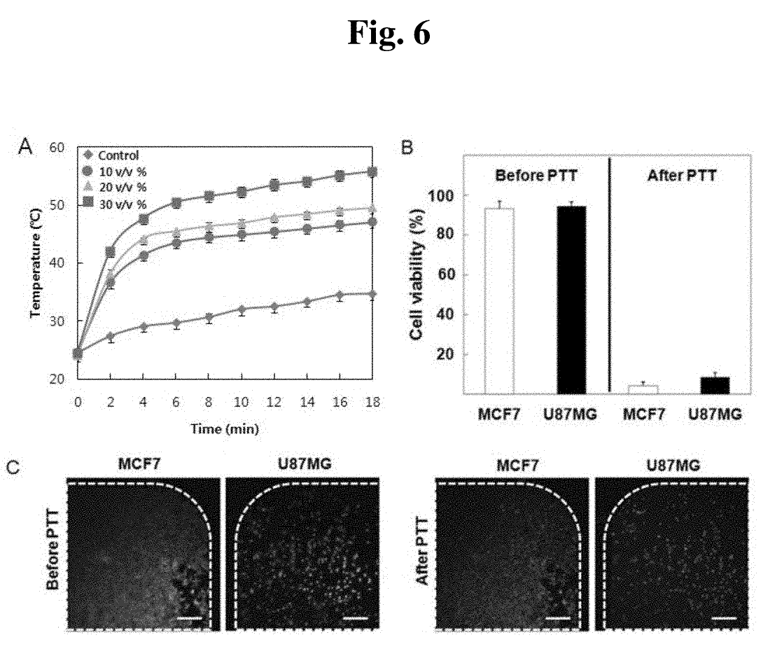

As a result of analyzing the temperature increase after 20, 30, and 40 μl of gold nanorods were mixed with 200 μl of cell culture medium and NIR laser was irradiated, the temperature increase was dependent on the concentration of gold nanorods (FIG. 6A). It was verified that, in the solution (20 v/v %), in which 30 μl of gold nanorods were mixed in 200 μl of the cell culture medium, the cells were killed by the photothermal effect while the shape of the cells was not influenced.

From the preliminary test results, when treated with 200 μl+40 μl of gold nanorods solution, the cells became unhealthy before the photothermal treatment (data not shown). Generally, the photothermal treatment at 45° C. or higher may damage tissues as well as cells. Therefore, the 200 μl+40 μl of gold nanorods were determined to be inappropriate in optimizing photothermal conditions.

Meanwhile, the glioblastoma cells and breast cancer cells were cultured in the chip for 1 day, and, in a similar manner, the cells were irradiated with NIR laser and analyzed by the live/dead assay, and as a result, most cells were dead by the photothermal effect.

### Co-Culture of Cancer Cells in 3D Microfluidic Device

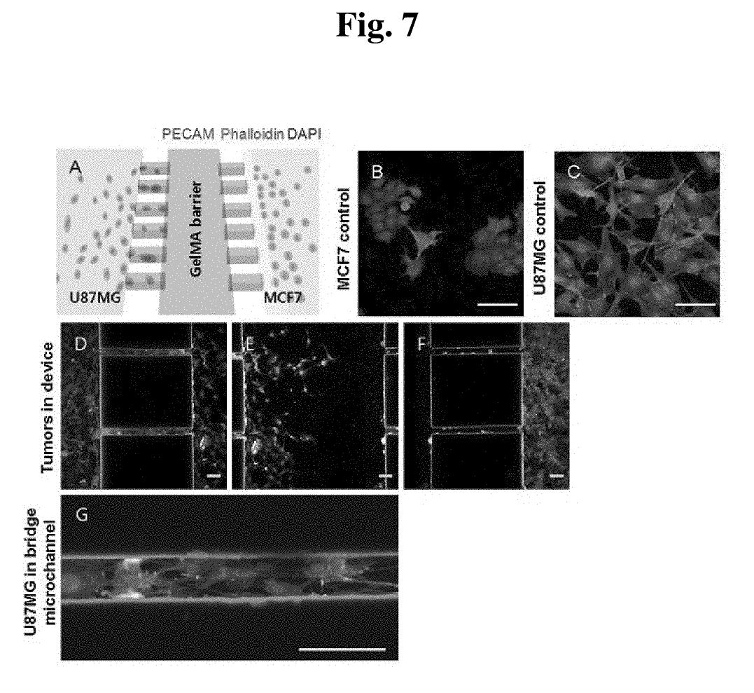

The glioblastoma cells and breast cancer cells were injected into different microchambers and co-cultured. The vascular endothelial cells were encapsulated in gelatin methacrylate hydrogels and injected into the cruciform microfluidic channel. The GelMA hydrogel injected into the microfluidic channel became a physical barrier, and the cross-contamination of the respective cancer cells and the culture media thereof did not occur. The vascular endothelial cell culture medium containing VEGF was allowed to flow through the microfluidic channel, and as a result, it was verified that the cancer cells (U87MG) migrated toward the vascular endothelial cells (FIG. 7).

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### REFERENCES

1. Madajewicz S, Chowhan N, Tfayli A, Roque C, Meek A, Davis R, Wolf W, Cabahug C, Roche P, Manzione J et al: Cancer 2000, 88(10):2350-2356.
2. Kesari S: Seminars in oncology 2011, 38 Suppl 4:S2-10.
3. Furnari F B, Fenton T, Bachoo R M, Mukasa A, Stommel J M, Stegh A, Hahn W C, Ligon K L, Louis D N, Brennan C et al: Genes & development 2007, 21(21):2683-2710.
4. Gupta P B, Kuperwasser C, Brunet J P, Ramaswamy S, Kuo W L, Gray J W, Naber S P, Weinberg R A: Nature genetics 2005, 37(10):1047-1054.
5. Miles F L, Pruitt F L, van Golen K L, Cooper C R: Clinical & experimental metastasis 2008, 25(4):305-324.
6. Piccirillo S G, Reynolds B A, Zanetti N, Lamorte G, Binda E, Broggi G, Brem H, Olivi A, Dimeco F, Vescovi A L: Nature 2006, 444(7120):761-765.
7. Fang J, Chen Y C: Current pharmaceutical design 2013, 19(37):6622-6634.
8. Cobley C M, Chen J, Cho E C, Wang L V, Xia Y: Chemical Society reviews 2011, 40(1):44-56.
9. Stone J, Jackson S, Wright D: Wiley interdisciplinary reviews Nanomedicine and nanobiotechnology 2011, 3(1):100-109.
10. Alkilany A M, Thompson L B, Boulos S P, Sisco P N, Murphy C J: Advanced drug delivery reviews 2012, 64(2):190-199.
11. Letfullin R R, Joenathan C, George T F, Zharov V P: Nanomedicine 2006, 1(4):473-480.
12. Terentyuk G S, Maslyakova G N, Suleymanova L V, Khlebtsov N G, Khlebtsov B N, Akchurin G G, Maksimova I L, Tuchin V V: Journal of biomedical optics 2009, 14(2):021016.
13. von Maltzahn G, Park J H, Agrawal A, Bandaru N K, Das S K, Sailor M J, Bhatia S N: Cancer research 2009, 69(9):3892-3900.
14. Zhang Z, Wang L, Wang J, Jiang X, Li X, Hu Z, Ji Y, Wu X, Chen C: Advanced materials 2012, 24(11):1418-1423.
15. Lee J M, Kim J E, Borana J, Chung B H, Chung B G: Electrophoresis 2013, 34(13):1931-1938.
16. Chung B G, Lin F, Jeon N L: Lab on a chip 2006, 6(6):764-768.
17. Nagrath S, Sequist L V, Maheswaran S, Bell D W, Irimia D, Ulkus L, Smith M R, Kwak E L, Digumarthy S, Muzikansky A et al: Nature 2007, 450(7173):1235-1239.
18. Hyun K A, Kwon K, Han H, Kim S I, Jung H I: Biosensors & bioelectronics 2013, 40(1):206-212.
19. Sung J H, Kam C, Shuler M L: Lab on a chip 2010, 10(4):446-455.
20. Chung S, Sudo R, Mack P J, Wan C R, Vickerman V, Kamm R D: Lab on a chip 2009, 9(2):269-275.
21. Shin Y, Han S, Jeon J S, Yamamoto K, Zervantonakis I K, Sudo R, Kamm R D, Chung S: Nature protocols 2012, 7(7):1247-1259.

1. A microfluidic chip for co-culture of cancer cells comprising:
(a) one or more microchambers as cell culture sections, including sample inlets;
(b) bridge channels connected to the microchambers; and
(c) a microfluidic channel connected to the bridge channels and including a hydrogel inlet,
wherein the microfluidic chip comprises a barrier formed by hydrogels and vascular endothelial cells,
wherein the hydrogels comprise gelatin-acryl polymer prepared by mixing gelatin and an acryl polymer,
wherein the hydrogels and the vascular endothelial cells are injected through the hydrogel inlet.

2. The microfluidic chip of claim 1, wherein the acryl polymer is selected from the group consisting of an acrylate and methacrylate copolymer, a methacrylate copolymer, a methyl methacrylate copolymer, an ethoxyethyl methacrylate copolymer, a cyanoethyl methacrylate copolymer, an aminoalkyl methacrylate copolymer, a poly(acrylate) copolymer, a polyacrylamide copolymer, a glycidyl methacrylate copolymer and a mixture thereof.

3. The microfluidic chip of claim 1, wherein the hydrogels comprise a 5-15 wt % concentration of gelatin-acryl polymer.

4. The microfluidic chip of claim 1, wherein the gelatin and the acryl polymer are photo-crosslinked in the hydrogels.

5. The microfluidic chip of claim 1, wherein the microfluidic chip is fabricated by using a polymer material selected from the group consisting of poly(dimethylsiloxane) (PDMS), polymethylmethacrylate (PMMA), polyacrylates, polycarbonates, polycyclic olefins, polyimides and polyurethanes.

6. The microfluidic chip of claim 1, wherein the microfluidic chip is joined to an upper portion of a plate facilitating optical measurement, which is selected from the group consisting of slide glass, crystal and glass.

7. The microfluidic chip of claim 1, wherein the microchambers are arranged in one or more columns and one or more rows.

8. A method for cell co-culture comprising:
(a) preparing a microfluidic chip for cell co-culture, comprising:
(i) one or more microchambers as cell culture sections, including sample inlets;
(ii) bridge channels connected to the microchambers; and
(iii) a microfluidic channel connected to the bridge channels and including a hydrogel inlet;
(b) preparing hydrogels that comprise gelatin-acryl polymer prepared by mixing gelatin and an acryl polymer;
(c) injecting hydrogels and vascular endothelial cells into the hydrogel inlet,
(d) inducing photo-crosslinking to construct a barrier; and
(e) injecting cancer cells into the sample inlets, followed by culturing.

9. A method for analyzing a photothermal therapy effect on cancer cells comprising:
(a) preparing a microfluidic chip for cell co-culture comprising:
(i) one or more microchambers as cell culture sections including sample inlets;
(ii) bridge channels connected to the microchambers; and
(iii) a microfluidic channel connected to the bridge channels and including a hydrogel inlet;
(b) preparing hydrogels that comprise gelatin-acryl polymer prepared by mixing gelatin and an acryl polymer;
(c) injecting the hydrogels and vascular endothelial cells into the hydrogel inlet,
(d) inducing photo-crosslinking to construct a barrier;
(e) injecting cancer cells through the sample inlets, followed by culturing;
(f) injecting nanoparticles exhibiting a photothermal effect through the sample inlets, followed by culturing; and
(g) irradiating a laser to the microchambers to analyze the extent of survival or death of the cancer cells.

10. The method of claim 9, wherein the nanoparticles are gold nanorods.
